(19) United States
(12) Patent Application Publication
(10) Pub. No.: US 2013/0261509 A1
(43) Pub. Date: Oct. 3, 2013

(54) DEVICE AND METHOD TO THERMALLY AFFECT DELIMITED REGIONS OF THE BODY OF A PATIENT

(71) Applicants: Patrick Gross, Buckenhof (DE); Clifford Weiss, Baltimore, MD (US)

(72) Inventors: Patrick Gross, Buckenhof (DE); Clifford Weiss, Baltimore, MD (US)

(21) Appl. No.: 13/899,984

(22) Filed: May 22, 2013

Related U.S. Application Data
(62) Division of application No. 12/947,259, filed on Nov. 16, 2010.

(30) Foreign Application Priority Data
Nov. 16, 2009 (DE) 102009053524.1

(51) Int. Cl. A61N 7/02; A61M 5/178

(52) U.S. Cl. CPC A61N 7/022; A61M 5/178
USPC 601/3

(57) ABSTRACT

Device to thermally affect delimited regions of the body of a patient, wherein it possesses at least one heating or cooling means that can be inserted into a blood vessel of the patient or can be attached to a blood vessel of the patient in order to heat or cool the blood in a portion of the blood vessel that leads into or away from a tissue to be treated.

### RELATED APPLICATION

The present application is a divisional application of Ser. No. 12/947,259, filed on Nov. 16, 2010.

### BACKGROUND OF THE INVENTION

1. Field of the Invention

The invention concerns a device to thermally affect delimited regions of the body of a patient.

2. Description of the Prior Art

In principle, many devices are known in order to supply or extract heat from the human body. In the domestic field, for instance, a hot-water bottle for heating or a washcloth wetted with cold water to cool burns are known by everyone. In the medical field additional devices are known that act more or less at a point. The introduction or removal of heat is thereby concentrated to a specific tissue that should be obliterated by the thermal effect. For example, liquid nitrogen as a cryogenic agent can be dabbed on a wart, causing the wart to be frozen and obliterated. It is also known to remove liver spots by means of a laser.

In addition to the possibility of affecting the surface of the body in the known examples, the possibility also exists to concentrate the introduction or removal of heat at tissue situated further inside the body. High-intensity focused ultrasound (HIFU) can be used for such introduction of heat, and a cryoablation catheter can be used for introduction of cold. In the case of devices acting at a point, the problem exists that blood vessels that are present in the region of the tissue to be treated or travel towards or away from this always provide for a heat dissipation or introduction of heat. The effect of the thermally operating device is thereby reduced. Furthermore, the danger exists of tissue that is not actually affected by the thermally operating device being damaged by heat dissipation or heat removal by means of the blood in the blood vessel that leads away from the tissue to be treated.

### SUMMARY OF THE INVENTION

An object of the invention is to provide a device with which the thermal effect on delimited regions of the body of a patient is improved.

This object is achieved by a device of the aforementioned type wherein, according to the invention, at least one heating or cooling arrangement is provided that can be inserted into a blood vessel of the patient or can be attached to a blood vessel of the patient in order to heat or to cool (i.e. to modify the temperature of) the blood in a portion of the blood vessel that leads toward or away from tissue to be treated.

The heating or cooling arrangement in or on the blood vessel considerably extends the possibilities for thermal effect. The heating or cooling arrangement can be used as a single device in order to affect the tissue to be treated. However, the heating or cooling arrangement can also be used in cooperation with an additional device to intensify the thermal effect of this additional device on the tissue to be treated or to reduce the effect of the additional device on the surrounding tissue.

For example, tissue to be treated can be affected by the introduction of heat. A heating arrangement that warms the blood surrounding the heating arrangement can then be inserted into or onto a blood vessel leading toward the tissue to be treated. The blood heated in such a manner then flows into the tissue to be treated, so this tissue is thermally affected. The heating arrangement can be provided in addition to a thermal ablation device that is present anyway and that likewise introduces heat into the tissue to be treated. However, the blood vessel leading away from the tissue to be treated again dissipates the introduced heat. In order to avoid damage to the downstream tissue by the discharged heat, a cooling arrangement that removes heat from the blood again can accordingly be provided in or on the blood vessel leading away from the tissue to be treated. Independent of the presence of a heating arrangement, the cooling arrangement can be used in cooperation with a thermal ablation device introducing heat.

Naturally, an opposite design (in terms of the thermal effect) is also possible wherein a cooling arrangement is inserted into or onto the blood vessel leading toward the tissue to be ablated and/or an additional thermal ablation device operating with heat extraction is used that acts directly on the tissue to be treated and/or a heating arrangement is provided in or on the a blood vessel leading away from the tissue to be treated. If more than one blood vessel leads toward or away from the tissue to be treated, more heating or cooling arrangements can accordingly be used.

The device can advantageously include an extracorporeal ultrasound device and a device to introduce gas bubbles into the vascular system of a person, wherein the gas bubbles are used as a heating arrangement by interacting with the extracorporeal ultrasound device.

The device can be fashioned as a syringe and the gas bubbles can consist of carbon dioxide. The ultrasound device is fashioned as a HIFU unit, wherein an introduction of heat inside the human body is already enabled. However, in this embodiment the heat introduction does not take place in the tissue to be heated itself; rather, the blood vessel leading towards the tissue to be treated is heated. In contrast to the conventional usage, the energy introduction ensues with a lower energy density so that the blood vessel is not ablated or, respectively, obliterated. Even without the effect of the gas bubbles, the blood located in the blood vessel that is in the field of view of the HIFU unit is heated. The effect of the HIFU on the blood is further intensified via the introduction of the gas bubbles, which are also known as what are called microbubbles and normally serve as a contrast agent for the ultrasound imaging. The interaction of the HIFU with the gas bubbles is linked with multiple advantages.

The gas bubbles can be introduced into the vascular system of the patient at any accessible point and also distribute in the entire body via the bloodstream. After traversing the bloodstream they are expelled again via the lungs. However, they are located within the vascular system in the body itself. In contrast to this, the HIFU acts on a spatially delimited region, wherein the effect cannot be limited to the blood of a vessel. Rather, given an introduction of heat into the blood of a vessel an effect on the vessel tissue is also to be included. The blood in the blood vessel that leads towards the tissue to be treated can accordingly not be so significantly heated as would be desirable. However, the gas bubbles in the blood are excited to vibrate by the HIFU, whereby the blood can be more significantly heated than by the use of HIFU alone. However, since the gas bubbles are only present in the blood, only the blood itself is more significantly heated and not the vessel wall in the heated area. Significantly heated blood then flows into the tissue to be treated (for example a tumor), whereby the tumor is obliterated by the introduction of heat.

Through the combination of the HIFU with the gas bubbles it is possible to heat the blood in the blood vessel (which the HIFU affects) more significantly than the surrounding tissue, whereby a concentrated introduction of heat into the tissue to be treated also occurs. The heating effect of the HIFU is thereby naturally directed toward the blood vessel region directly before the tissue to be treated and not, for instance, at a far distance from this. The heat introduction can be better limited to the tissue to be treated—in particular in the case of a tumor—than via the direct thermal ablation of the tissue to be treated by means of the HIFU.

In a further embodiment, the device can be fashioned as a cuff for attachment to the body of a patient, wherein the cuff has a heating or cooling device that heats or cools a portion of the part of the body that is covered by the cuff. For thermal interaction with a blood vessel it is not necessary that the heating or cooling arrangement is located in the human body itself. In particular given blood vessels that run directly below the surface of the skin, the insertion of the heating or cooling means into the human body can be avoided. In contrast to a hot-water bottle that introduces heat into the body over a large area and in a non-specific manner, given the cuff it is provided that the heating or cooling device heats or cools only a portion of the part of the body of the patient that is covered by the cuff. Inside the body a blood vessel whose blood is heated or cooled by means of the heating or cooling device is also accordingly located in the heated or cooled region.

To adapt to different body sizes and/or body circumferences, the cuff can be inflatable and consist of an expandable material. The cuff can also have two ends and be reversibly sealable at these by means of a hook-and-loop fastener or another device. The cuff, adapted to the respective body part, can enclose any arbitrary part of the body, for example a leg, a finger, the stomach, the ribcage, the throat or an arm. The adaptability of the cuff makes it possible to attach it to patients of various shapes.

In a further embodiment, the device can be fashioned as a cuff for arrangement on a blood vessel in the body of a patient, wherein the cuff possesses a heating or cooling device that is fashioned to heat or cool at least a portion of the blood in the blood vessel that is enclosed by the cuff. Not all blood vessels can be reached via cuffs that are to be applied outside the body so that a thermal interaction between the blood in the blood vessel and a heating or cooling device an occur at or in the cuff without damaging the intervening tissue. In this case the cuff is to be moved closer to the blood vessel. The cuff can possess a heatable resistor for heating and a Peltier cooler for cooling. The cuff itself can be designed so as to be thermally insulated against the outside in order to limit the heat exchange in the direction of the blood vessel.

In a further embodiment, the device can be fashioned as a catheter for insertion into the blood vessel of a patient, wherein the catheter possesses a heating or cooling device that is fashioned to heat or cool the blood that surrounds the catheter in the blood vessel. The catheter is advantageously already located in the blood vessel and accordingly comes into direct contact with the blood surrounding it. A more direct heat exchange between the blood and the catheter can thereby occur. The heating or cooling arrangement can act at a point; but the heat exchange can also be fashioned as a point-shaped heating or cooling source over a larger area. In particular, the entire distal end of the catheter can act in a heating or cooling manner on the surrounding blood. The heat exchange is limited to a portion of the blood in the blood vessel even given a larger-area design of the heat exchanging region of the catheter.

Corresponding spacing devices can be mounted on the catheter to separate the catheter from the vessel well. For example, uniformly distributed inflatable balloons can be provided in the circumferential direction that are inflated if the catheter is located in the final position, and thereby separate the catheter from the vessel wall.

The catheter can advantageously possesses a heatable resistor for heating or a Peltier cooler for cooling. The heatable resistor can represent both a rather point-shaped heat source and be wound externally around the catheter over a larger area in order to be able to produce the heat exchange with the surrounding blood at a larger area. The catheter can also simultaneously possess a Peltier cooler and a heatable resistor that are operated depending on the use location and use case of the catheter.

Alternatively or additionally, the catheter can possess at least one lumen into which a medium—in particular a liquid or a gas—can be introduced, wherein the medium can be used for heating or cooling. Since a heat exchange is reasonable only in the region of the digital end of the catheter, the lumen is largely to be thermally insulated so that the heat exchange actually occurs only in the intended region. In particular, the catheter in the region of the distal end can consist of a material that optimally simplifies a heat exchange. The material that is used should possess a high coefficient of thermal conduction and nevertheless should be flexible. Metals and carbon nanotubes have good heat conduction capabilities.

Alternatively or additionally, the catheter can have a coil as well as a lumen with an opening, wherein iron-containing particles can be introduced into the blood vessel via the lumen, which iron-containing particles can be used to heat the blood surrounding the catheter. In comparison to an IVMRI catheter, the coil must be fashioned to generate stronger alternating magnetic fields. The blood containing iron-containing particles can thereby be heated.

All known iron-containing MR contrast agents suggest themselves in particular as iron-containing particles. These are also designated as SPIO (Superparamagnetic Iron Oxides) or USPIO (Ultrasmall Superparamagnetic Iron Oxide).

The invention also concerns a method to thermally affect a portion of the blood in a blood vessel of a patient. This is characterized in that a heating or cooling means is introduced into the blood vessel of the patient or is arranged at a blood vessel of the patient in order to heat or to cool the blood in a portion of the blood vessel that leads toward or away from a tissue to be treated. Any of the devices already described can be advantageously used to implement the method.

### BRIEF DESCRIPTION OF THE DRAWINGS

FIG. 1 shows an ultrasound device and a heating arrangement.

FIG. 2 shows an ultrasound device and a cooling arrangement.

FIG. 3 shows a catheter in a first embodiment.

FIG. 4 shows a catheter in a second embodiment.

FIG. 5 shows a catheter in a third embodiment.

FIG. 6 shows a catheter in a fourth embodiment.

FIG. 7 shows a cuff in a first embodiment.

FIG. 8 shows a cuff in a second embodiment.

FIGS. 9 through 12 show cross section views of a cuff.

### DESCRIPTION OF THE PREFERRED EMBODIMENTS

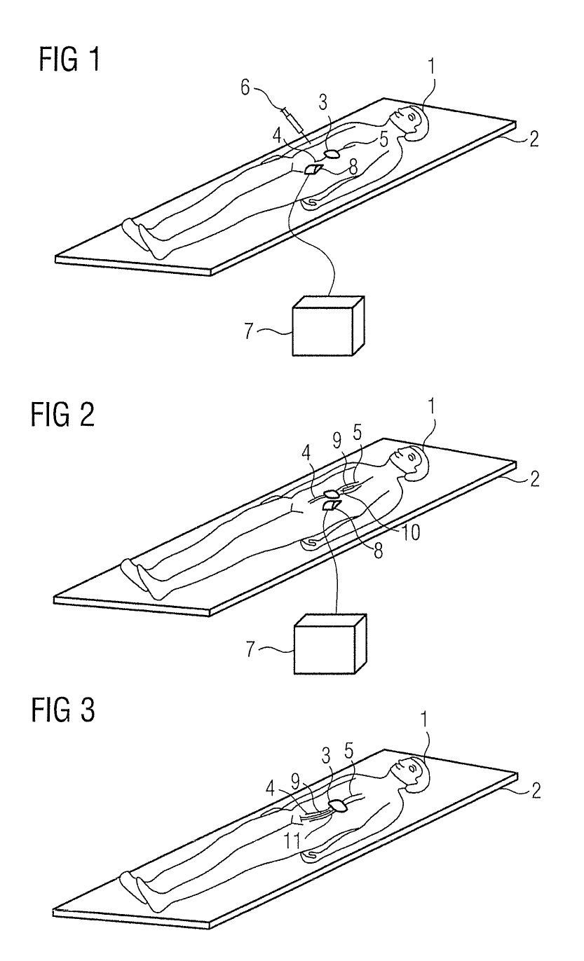

FIG. 1 shows a patient 1 on a patient bed 2. The tissue 3 to be treated in or on the body of the patient 1 can in principle be located at any points of the body; however, the device according to the invention and the method according to the invention are only used if a blood vessel leads either into the tissue 3 to be treated and/or away from the tissue 3 to be treated or at least runs through the surrounding region. A blood vessel 4 leading toward said region is understood as a blood vessel for which blood therein is completely encompassed in the tissue 3 to be treated. If a blood vessel 4 leading toward the tissue 3 to be treated is present, a blood vessel 5 leading away naturally also exists.

It is known to obliterate tissue inside a patient 1 by means of HIFU. HIFU-capable ultrasound devices 7 possess an ultrasound body 8 with which the field distribution of the sound can be manipulated so that a maximum energy injection results at a predetermined location inside the body. The tissue 3 to be treated is directly obliterated in this way.

However, this procedure is problematic if a blood vessel 4 leading to the tissue 3 to be treated and a blood vessel 5 leading away exist. On the one hand the tissue 3 to be treated is cooled by these vessels; on the other hand the heat energy introduced into the tissue 3 to be treated is transported away into the tissue surrounding the outgoing blood vessel 5. Therefore the ultrasound head 8 is not used heat the tissue 3 to be treated but rather to heat the blood in the blood vessel 4 leading in. The heating of the blood vessel 4 leading in is achieved without damaging the surrounding tissue and the vessel wall in that gas bubbles are injected into the vascular system of the patient 1 by means of a syringe 6. Depending on the location at which the gas bubbles were introduced into the vascular system, these distribute in the body of the patient. However, a heating of the blood via the gas bubbles only occurs where ultrasonic waves interact with the gas bubbles. The location of the interaction can accordingly be established via the positioning of the ultrasound head 8. The ultrasonic waves that are generated by means of the ultrasound device 7 produce a vibration of the gas bubbles and thereby a heating of the blood surrounding the gas bubbles. Gas bubbles that are set into vibration by ultrasonic waves and can be applied without damaging the vascular system of the patient are sufficiently known. For example, what are known as microbubbles can be used for this that normally serve as an ultrasound contrast agent. Differences between the individual embodiments exist in the envelope and in the gas core. Microbubbles with a lipid-galactose envelope and an air filling are known under the trade name “Levovist”. However, there are also microbubbles with an albumin envelope and a core consisting of octafluoropropane.

The ultrasound head 8 is aligned and set so that an interaction occurs with the gas bubbles in the portion of the incoming blood vessel 4 that directly borders the region 3 to be treated. The heated blood thus enters directly into the region 3 to be treated without exchanging heat with other tissue beforehand. This arrangement furthermore enables the energy injection to be concentrated on the blood containing the gas bubbles and bombarded with the ultrasound head 8, which is why an unnecessary heating and corresponding damage to the surrounding tissue can be avoided.

FIG. 2 shows a further exemplary embodiment with a HIFU in which this directly heats the tissue 3 to be treated. To protect the tissue adjoining the vessel 5 leading away, a catheter 9 is inserted into this outgoing blood vessel 5. This has at the distal end a Peltier cooler 10 with which the surrounding blood is cooled. In order to avoid a placement of the Peltier cooler 10 on the vessel wall of the outgoing blood vessel 5, spacers can be provided at the catheter 9. For example, these can be realized as inflatable balloons, wherein these balloons are arranged inside the catheter 9 in the unfilled state. The balloons are filled via lumens and are distributed uniformly on the catheter 9 in the circumferential direction. The balloons are to be arranged near to the Peltier cooler 10; the catheter or, respectively, the distal region of the catheter 9 is accordingly held in the center of the outgoing vessel 5. The balloons can also be used as spacers in the following embodiments of the catheter 9.

In a further embodiment it is also possible to use the catheter 9 as a heating means. For this the catheter 9 is positioned in the incoming blood vessel 4 so that the distal end is in immediate proximity to the tissue 3 to be treated. A heatable resistor 11 with which the blood surrounding the catheter tip can be heated is located at the tip or in immediate proximity to the tip of the catheter 9. The heat dissipation ensues in a large-surface area around the catheter tip in order to enable a uniform heating of the surrounding blood.

In addition to the catheter 9, the ultrasound device 7 can be used as a heating means. Instead of or in addition to the ultrasound device 7, an additional catheter 9 as a cooling means can be arranged in the outgoing blood vessel 5. Different types can thus also be connected with one another to affect the tissue 3 to be treated and the blood in the blood vessels 4 and 5.

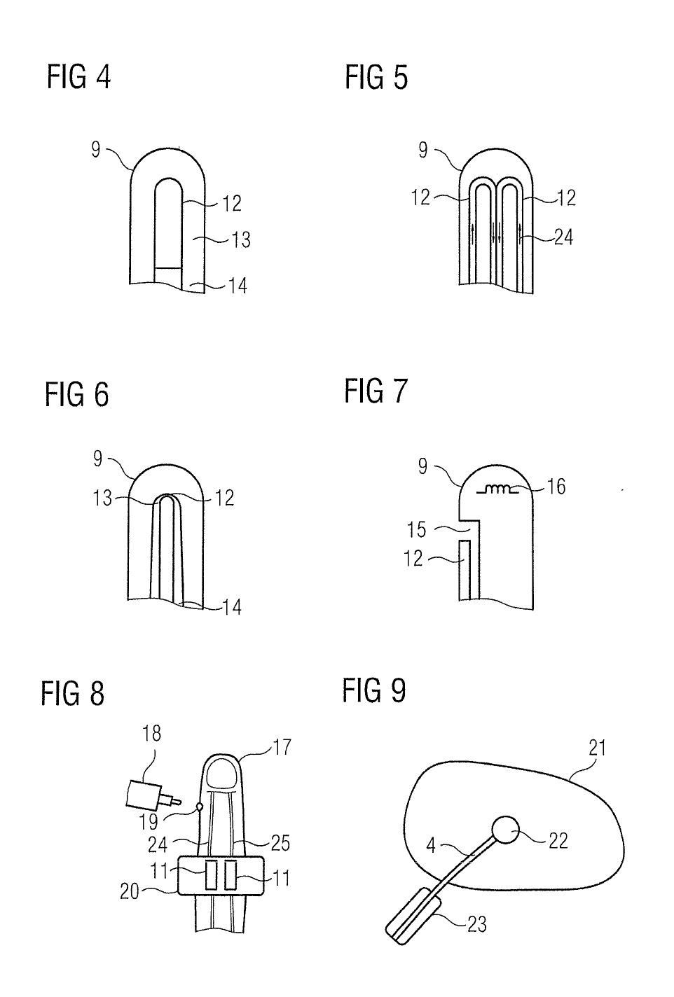

FIGS. 4-6 show additional embodiment possibilities of the catheter 9. FIG. 4 shows an embodiment in which the catheter can be used as a heating or also as a cooling means depending on the medium that is used. For this a lumen 12 that can be filled with a medium is located in the catheter 9. A liquid heated or cooled outside of the body is advantageously used as a medium. The lumen is ideally divided up into an uninsulated segment 13 and a thermally insulated segment 14 so that a heat exchange between the liquid in the lumen and the catheter occurs only in the region of the tip of the catheter. A large-area heat exchange over the entire tip of the catheter can ensue via the lumen 12.

Instead of a liquid, a gas or a liquid/gas mixture or a liquid with solid particles can also be used. Due to the low volume of the lumen 12, the heat capacity stored by the liquid is also relatively low. It is therefore advantageous if a chemical reagent that is capable of dispensing or extracting heat due to the chemical reaction is used as a liquid. In this case a greater heat capacity can be introduced into the blood or removed from the blood by the liquid than is possible via the one-time heating outside of the body.

In order to circumvent the problem of the too-low heat capacity of the medium in the lumen 12, multiple U-shaped lumens 12 can also be used in the catheter 9 as FIG. 6 shows. The lumens are arranged annularly and (due to the U-shape) have a region in which the medium travels towards the distal tip of the catheter 9 and a region in which the medium travels away from the tip. The incoming portion is respectively arranged on the outside while the outgoing portion is to be located inside the catheter. The flow direction of the medium is indicated by the arrows 24.

In this embodiment the lumens 12 also possess an uninsulated segment 13 and a thermally insulated segment 14 in order to limit the heat exchange to the region of the tip of the catheter 9.

FIG. 6 shows an additional catheter 9 with a lumen 12. The thermal insulation of the lumen 12 is thereby variable, which is represented by the varying thickness of the thermal insulation. This means that the lumen is not insulated at the tip of the catheter, possesses a slight insulation in the following region that becomes increasingly greater in the following segments. A heat exchange gradient can thereby be established that is reflected in a temperature gradient. The catheter is thus heated most significantly at the tip while the heating decreases with increasing distance from the tip. A heat exchange between the medium in the lumen 12 and the catheter 9 no longer occurs as of a predetermined distance from the tip.

In this way it is achieved that the blood that flows past the catheter 9 closer to the tissue 3 to be treated is more significantly heated than the blood that is further removed from the tissue 3 to be treated. This serves to protect the vessel wall in the region of the tip of the catheter 9.

In the embodiments described in FIGS. 4-6 the catheter can respectively be used as a heating or cooling means. The effect results solely from the temperature of the medium in the lumen 12.

In an alternative embodiment, the catheter 16 has an opening 15 via which a medium can be directly introduced into the blood. This medium contains iron-containing particles. Iron-containing particles that can be introduced harmlessly into the vascular system of the person are already known as contrast agents for MR imaging. In order to achieve a heating of the blood containing the iron-containing particles, a coil 16 is located in the tip of the catheter 9. This generates a magnetic field whereby the blood is heated via the iron particles. Multiple coils 16 can also be provided to apply specific predetermined gradient fields, wherein the gradient field generated as a whole is variable by feed current to the individual coils 16. Arbitrary gradient fields can be generated by means of three orthogonal coils 16.

In order to be able to heat the blood surrounding the catheter tip within a reasonable temperature range—i.e. by at least multiple degrees Celsius—the iron particles must be more highly concentrated in the medium 15 than would be the case given a use of the iron particles as a contrast agent for magnetic resonance imaging. Instead of one or more coils 16, the coils of a conventional magnetic resonance device can be used. In this case the catheter 9 serves merely to supply the iron-containing particles; the patient 1 is to be arranged in a magnetic resonance device to heat the particles. In this case the heating of the blood can be monitored simultaneously with temperature-sensing imaging magnetic resonance methods.

In addition to a use of a heating or cooling arrangement in the blood vessel itself, a heating or, respectively, cooling of the blood in the blood vessel from the outside is also possible. Here an additional differentiation is made to the effect that the heating or cooling arrangement can be arranged inside and outside the body.

FIG. 8 shows an embodiment of a of a heating means mounted outside of the body in the form of a cuff 20. This is directed around the finger 17 of the patient 1. A wart 18 that is frozen with a cooling device 19 is located on the finger 17. To protect the surrounding tissue in the finger 17 two heatable resistors 11 are located in the cuff 20, with which resistors 11 both the blood in the incoming blood vessel 24 and the blood in the outgoing blood vessel 25 are heated. The heating of both the incoming blood vessel 24 and the outgoing blood vessel 25 is reasonable in this case since the incoming blood vessel 24 does not lead directly to the region 3 to be treated (which in this case exists in the wart 19) but rather merely runs past this. Instead of two heatable resistors 11, the cuff 20 can also merely possess one heatable resistor 11 in order to merely heat the incoming blood vessel 24 or the outgoing blood vessel 25. The number of heatable resistors 11 thereby depends on the usage location of the cuff 20. In principle, a separate heatable resistor Ills to be provided for each blood vessel. Since the number and arrangement of blood vessels varies from person to person, it is therefore particularly useful to arrange a number of heatable resistors 11 distributed circumferentially on the cuff 20, which resistors 11 can be individually activated and thus specifically heated. It is thereby possible to heat the blood in individual blood vessels specifically without heating the entire finger 17 in a large volume.

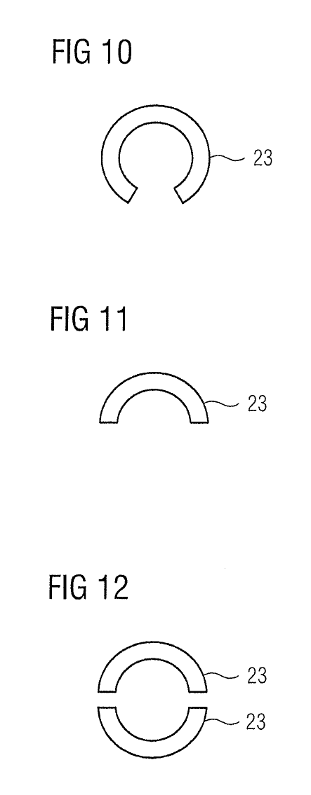

FIG. 19 shows a liver 21 in which is located a tumor 22. The blood vessel 4 around which a cuff 23 was placed leads to the liver 21. The cuff 23 can exhibit the most varied shapes in cross section in order to be able to be attached to different vessels. Such embodiments result from FIGS. 10-12.

FIG. 10 shows a cuff with a horseshoe shape while a semicircle shape arises from FIG. 11. As FIG. 12 shows, the connection of two semicircular cuffs can also be made for a coverage of the blood vessel 4 around its entire circumference. Independent of the embodiment of the cuff 23 in the individual case, this possesses a heatable resistor 11 and/or a Peltier cooler 10 in order to be able to heat or cool the blood in the incoming blood vessel 4. Depending on whether the tumor 22 should be obliterated by heat or frozen, the respective thermal effect can in principle be produced or supported with the cuff 23. In addition to the cuff 23, additional heating or cooling or, respectively, freezing devices can thus be used. For example, in addition to the cuff 23 a HIFU-capable ultrasound device 7 can be used, wherein the cuff 23 at the incoming vessel 7 heats the blood inside the vessel 4. Analogous to the embodiments already addressed with regard to the catheter 9, in this case a cuff 23 with a Peltier cooler 10 can also be arranged on the outgoing vessel 5.

Although modifications and changes may be suggested by those skilled in the art, it is the intention of the inventor to embody within the patent warranted hereon all changes and modifications as reasonably and properly come within the scope of his contribution to the art.

We claim as our invention:

1. A device to thermally affect delimited regions of the body of a patient, comprising:
at least one temperature modifying device configured for in vivo interaction with a blood vessel of a patient, said blood vessel leading into or away from tissue to be thermally treated; and
said at least one temperature modifying device being configured to modify the temperature of blood flowing in said blood vessel and comprising an extracorporeal ultrasound device and a gas bubble-introducer that introduces gas bubbles into the vascular system of said patient, said gas bubbles elevating said temperature in said blood in said blood vessel by interaction with said extracorporeal ultrasound device.

2. A device as claimed in claim 1 wherein said temperature modifying device is configured for insertion into said blood vessel.

3. A device as claimed in claim 1 wherein said temperature modifying device is configured for attachment to said blood vessel.

4. A device as claimed in claim 1 wherein said temperature modifying device comprises a syringe.

5. A device as claimed in claim 1 wherein said gas bubble introducer introduces gas bubbles of carbon dioxide into said blood in said blood vessel.

6. A method to thermally affect a portion of blood in a blood vessel of a patient, comprising the steps of:
designating a tissue region to be thermally treated in vivo in a patient, and identifying a blood vessel selected form the group consisting of blood vessels leading to said tissue and blood vessels leading away from said tissue;
forming a temperature-modifying device as an extracorporeal ultrasound device and a gas bubble-introducer that introduces gas bubbles into the vascular system of said patient, said gas bubbles elevating said temperature in said blood in said blood vessel by interaction with said extracorporeal ultrasound device;
introducing said temperature-modifying device in vivo into the patient to interact with blood in said blood vessel; and
operating said temperature-modifying device in vivo to modify the temperature of the blood in the blood vessel.
